(19) United States
(12) Patent Application Publication
(10) Pub. No.: US 2005/0079506 A1
(43) Pub. Date: Apr. 14, 2005

(54) Filled, biological microarray and method for use

(75) Inventors: Jeffrey W. Leon, Rochester, NY (US); Tiecheng A. Qiao, Webster, NY (US); Christine J. Landry-Coltrain, Fairport, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/682,271

(22) Filed: Oct. 9, 2003

(51) Int. Cl. C12Q 1/68; C12M 1/34

(52) U.S. Cl. 435/6; 435/287.2

(57) ABSTRACT

The present invention relates to a biological microarray element comprising a support having disposed thereon at least one layer comprising filler and gelatin, and at least one functional compound, wherein the functional compound comprises a first functional group capable of interacting with gelatin and a second functional group capable of interacting with a biological capture agent, wherein the first functional group is the same as or different from the second functional group. Also provided is a method of making a biological microarray element comprising providing a support; and coating a layer comprising filler and gelatin and at least one functional compound, wherein the functional compound comprises a first functional group capable of interacting with the gelatin and a second functional group capable of interacting with a biological capture agent, wherein the first functional group is the same as or different from the second functional group.

### FIELD OF THE INVENTION

The present invention relates to fabricating biological microarrays in general and in particular to a method that utilizes a filled, gelatin-based substrate wherein the gelatin substrate is modified to reduce background noise.

### BACKGROUND OF THE INVENTION

The completion of the Human Genome project spurred the rapid growth of a new interdisciplinary field of proteomics which includes identification and characterization of complete sets of proteins encoded by the genome, the synthesis of proteins, post-translational modifications, as well as detailed mapping of protein interaction at the cellular regulation level.

While 2-dimensional gel electrophoresis in combination with mass spectrometry still remains the dominant technology in proteomics study, the successful implantation and application of deoxyribonucleic acid (hereinafter referred to as DNA) microarray technology to gene profiling and gene discovery have prompted scientists to develop protein microarray technology and apply microchip based protein assays to the field of proteomics. For example, in WO 00/04382 and WO 00/04389, a method of fabricating protein microarrays is disclosed. A key element in the disclosure is a substrate consisting of a solid support coated with a monolayer of thin organic film on which protein or a biological capture agent can be immobilized.

Nitrocellulose membrane was widely used as a protein blotting substrate in Western blotting and Enzyme Linked Immunosorbent Assay (ELISA). In WO 01/40312 and WO 01/40803, antibodies are spotted onto a nitrocellulose membrane using a gridding robot device. Such spotted antibody microarrays on a nitrocellulose membrane substrate have been shown to be useful in analyzing protein mixtures in a large parallel manner.

WO 98/29736 describes an antibody microarray with an antibody immobilized onto a N-hydroxysuccinimidyl ester modified glass substrate. In U.S. Pat. No. 5,981,734 and WO 95/04594, a polyacrylamide based hydrogel substrate technology is described for the fabrication of DNA microarrays. More recently, in Anal. Biochem. (2000) 278, 123-131, the same hydrogel technology was further demonstrated as useful as a substrate for the immobilization of proteins in making protein microarrays.

In the above cited references, the common feature is the requirement of a solid support that allows covalent or non-covalent attachment of a protein or a biological capture agent on the surface of the support. In DNA microarray technology, a variety of surfaces have been prepared for the deposition of pre-synthesized oligos and polymerase chain reaction (PCR) prepared cDNA probes. For example, in EP 1 106 603 A2, a method of preparing vinylsulfonyl reactive groups on the surface to manufacture a DNA chip is disclosed. Even though the invention is useful in preparing DNA chips, it is not suitable for protein microarray applications. Unlike DNA, proteins tend to bind to surfaces in a non-specific manner and, in doing so, lose their biological activity. Thus, the attributes for a protein microarray substrate are different from those for a DNA microarray substrate in that the protein microarray substrate must not only provide surface functionality that is capable of interacting with biological capture agents, but must also resist non-specific protein binding to areas where no biological capture agents have been deposited.

A conventional way of generating biological attachment chemistry on a glass surface is to use silane coupling chemistry as described by Edwin P. Plueddemann, “Silane Coupling Agents” 2nd Ed., Plenum Press, New York, 1991, to graft the appropriate biological attachment chemistry onto a glass surface. To perform such grafting, a glass surface must be either plasma discharge treated or chemically treated with chemical reagents to provide a hydrophilic surface.

Bovine serum albumin (BSA) has been demonstrated to be a useful reagent in blocking proteins from non-specific surface binding. Polyethylene glycol and phospholipids have also been used to passivate surfaces and provide a surface, which is resistant to non-specific binding. However, all of these methods suffer disadvantages either because surface preparation takes a long time or because the method of surface modification is complex and difficult, making the method less than an ideal choice for large scale industrial manufacture.

US application publication 2003/0138649 and U.S. application Ser. No. 10/091,644, describe a low cost method of making protein microarray substrates using a gelatin coating to create a reactive surface for immobilization of biological capture agents. The gelatin modified surface effectively eliminates non-specific protein binding and the dimensionally stable substrate with chemical functionality for the immobilization of biological capture agents has sufficient adhesive strength on its surface to bind the coated gelatin layer so that the gelatin layer does not frill, when the coated substrate is wet during any biological processing, or stripping, when the coated substrate is dry. However, there remains a problem with the inherent flourescence of the microarray substrate material, which provides excessive background noise.

### PROBLEM TO BE SOLVED

There remains a need for a biological microarray element, which provides a dimensionally stable gelatin-coated substrate with chemical functionality for the immobilization of biological capture agents, also referred to herein as tags, bioaffinity tags, or bio-tags, and lower inherent fluorescence, resulting in reduced background noise.

### SUMMARY OF THE INVENTION

The present invention relates to a biological microarray comprising a support having disposed thereon at least one layer comprising filler and gelatin, and at least one functional compound, wherein the functional compound comprises a first functional group capable of interacting with gelatin and a second functional group capable of interacting with a biological capture agent, wherein the first functional group is the same as or different from the second functional group.

Also provided is a method of making a biological microarray element comprising providing a support; and coating a layer comprising filler and gelatin and at least one functional compound, wherein the functional compound comprises a first functional group capable of interacting with the gelatin and a second functional group capable of interacting with a biological capture agent, wherein the first functional group is the same as or different from the second functional group.

### ADVANTAGEOUS EFFECT OF THE INVENTION

The present invention includes several advantages, not all of which are incorporated in a single embodiment. The invention is particularly useful in fabricating biological microarrays, providing a substrate with functionalities capable of interacting specifically with biological capture agents immobilized on its surface and that is substantially resistant to non-specific binding. In addition, substrates prepared with filler, gelatin and a functional compound may require a very low concentration of biological sample in fabricating biological microarrays when compared with unmodified gelatin substrates. The gelatin substrates of the invention can be readily manufactured at low cost. The usefulness of the claimed substrate for biological attachment is demonstrated below in the examples, using several chemical modification methods and Enzyme Linked Immunosorbent Assay (ELISA). The present invention also demonstrates reduced background noise, for example, lowered background florescence, without sacrificing the immobilization capacity of the biological capture agent.

### BRIEF DESCRIPTION OF THE DRAWINGS

FIG. 1 is a structure of vinylsulfone and vinylsulfone-containing precursor “H” monomers.

### DETAILED DESCRIPTION OF THE INVENTION

The present invention relates to an array of biological capture agents, usually antibodies, on a support coated with gelatin. The inherent fluorescence of the gelatin can be significantly reduced by incorporating one or more filler species within the gelatin layer.

Supports of choice for biological microarray applications may be organic, inorganic or biological. Some commonly used support materials may include glass, plastics and polymers, metals, and semiconductors. The support may be transparent or opaque, flexible or inflexible. In some cases, the support may be a porous membrane, for example, nitrocellulose and polyvinylidene difluoride. Opaque supports include plain paper, coated paper, resin-coated paper such as polyolefin-coated paper, synthetic paper, photographic paper support, melt-extrusion-coated paper, and polyolefin-laminated paper, such as biaxially oriented support laminates. Biaxially oriented support laminates are described in U.S. Pat. Nos. 5,853,965, 5,866,282, 5,874,205, 5,888,643, 5,888,681, 5,888,683, and 5,888,714, the disclosures of which are hereby incorporated by reference. These biaxially oriented supports include a paper base and a biaxially oriented polyolefin sheet, typically polypropylene, laminated to one or both sides of the paper base. The support can also consist of microporous materials such as polyethylene polymer-containing material sold by PPG Industries, Inc., Pittsburgh, Pa. under the trade name of Teslin®, Tyvek® synthetic paper (DuPont Corp.), impregnated paper such as Duraform®, and OPPalyte® films (Mobil Chemical Co.) and other composite films listed in U.S. Pat. No. 5,244,861. Transparent supports include glass, cellulose derivatives, such as a cellulose ester, cellulose triacetate, cellulose diacetate, cellulose acetate propionate, cellulose acetate butyrate, polyesters, such as poly(ethylene terephthalate), poly(ethylene naphthalate), poly-1,4-cyclohexanedimethylene terephthalate, poly(butylene terephthalate), and copolymers thereof, polyimides, polyamides, polycarbonates, polystyrene, polyolefins, such as polyethylene or polypropylene, polysulfones, polyacrylates, polyether imides, and mixtures thereof. The papers listed above include a broad range of papers, from high end papers, such as photographic paper to low end papers. The term as used herein, “transparent” means the ability to pass radiation without significant deviation or absorption. However, to improve robustness and reproducibility, it is more desirable to use a solid support that has dimensional stability.

Glass, or fused silica, is the most commonly used microarray support in the art. Generally, a glass support is planar, and has high flatness and clarity. Preferably, the glass does not fluoresce, and has a thickness from 0.1 mm to 5 mm. The glass support may have any dimensions and may be cut into various sizes according to its intended uses.

In another embodiment of the invention, a polymeric support may be coated with the filled gelatin layer. Typical polymeric supports which form supporting surfaces for use with this invention include cellulose esters such as cellulose nitrate and cellulose acetate, poly(vinyl acetal) polymers, polycarbonates, polyesters such as polymeric, linear polyesters of bi-functional saturated and unsaturated aliphatic and aromatic dicarboxylic acids condensed with bi-functional polyhydroxy organic compounds such as polyhydroxy alcohols, for example, polyesters of alkylene glycol and/or glycerol with terephthalic, isophthalic, adipic, maleic, fumaric and/or azelaic acid, polyhalohydrocarbons such as polyvinyl chloride, and polymeric hydrocarbons, such as polystyrene and polyolefins, particularly polymers of olefins having from 2 to 20 carbon atoms.

The support used in the invention may have a thickness of from 50 micrometers to 5 millimeters, preferably from 0.5 to 1.5 millimeters. These supports may be used alone or may be utilized as coatings on metal, glass, and other solid surface. It is preferred that the support has substantial dimensional stability when wet.

Gelatin has been used in the photographic industry as a binder for various chemical components, and the process of making high quality gelatin is well established in industry. Because gelatin is made of biological materials, it is biologically compatible with biological capture agents on the microarray. The gelatin-coated surface provides a biologically benign surface for the immobilization of biological capture agents onto the microarray. Gelatin may also render a surface that substantially reduces background noise as a result of non-specific binding. More optimally, a filler, added to the gelatin, will reduce the amount of inherent fluorescence derived from the gelatin still further, which manifests itself as additional background noise reductions.

Normally, gelatin is coated onto a support and gelation occurs through a process by which gelatin solutions or suspensions of gelatin and other materials form continuous three-dimensional networks that exhibit no steady state flow. This can occur in polymers by polymerization in the presence of polyfunctional monomers, by covalent cross-linking of a dissolved polymer that possesses reactive side chains and by secondary bonding, for example, hydrogen bonding, between polymer molecules in solution. Polymers such as gelatin exhibit thermal gelation which is of the latter type. The process of gelation or setting is characterized by a discontinuous rise in viscosity. (See, P. I. Rose, “The Theory of the Photographic Process”, 4^{th} Edition, T. H. James ed. pages 51 to 67).

There are two types of gelatin: acid pretreated and alkaline pretreated. The preferred gelatin is alkaline pretreated gelatin from bovine bone, but gelatin may also come from other sources. Examples include, but are not limited to, pig gelatin, fish gelatin and fowl gelatin.

The amount of gelatin used should be sufficient to impart cohesive strength to the element, and should also be in an amount sufficient such that there are essentially no interstitial voids in the layers. In a preferred embodiment of the invention, the gelatin is rich in amine moieties and is present in an amount of from 15 to 99% by weight, and most preferably, in an amount from 20 to 75% by weight of each layer.

The fillers used in the present invention may be broken down into three main classes: inorganic particles, organic particles, or soluble polymers. These classes of fillers may also be used in combination.

Organic particles may preferably be polymeric materials. The polymeric materials may be of any class of synthetic or non-synthetic polymers, provided that they are water-insoluble and can be prepared in a particulate form which can preferably be dispersed in water or in a water-miscible carrier solvent. Polymer classes may include, but are not necessarily limited to addition polymers, poly (alkylene oxides), cellulosics, phenol-formaldehyde polymers, urea-formaldehyde polymers and condensation polymers consisting of one or more of the following repetitive units: esters, amides, imides, carbonates, urethanes, and ethers. The polymer particles may be coalescing or non-coalescing and may be of any morphology (core-shell, solid, porous).

In one embodiment, the organic filler particles may be monodisperse or relatively monodisperse. “Monodisperse” means that the coefficient of variation of the particle size distribution, that is, the standard deviation as a percentage of the mean, will be less than 20%. Preferably, the coefficient of variation will be less than 15%. Most preferably, the coefficient of variation will be less than 10%.

Preferably this polymer will be an addition polymer of monomers containing α,β-ethylenic unsaturation, which have limited solubility in water. These include, but are not necessarily limited to methacrylic acid esters, such as methyl methacrylate, ethyl methacrylate, isobutyl methacrylate, 2-ethylhexyl methacrylate, benzyl methacrylate, phenoxyethyl methacrylate, cyclohexyl methacrylate and glycidyl methacrylate, acrylic/acrylate esters such as methyl acrylate, ethyl acrylate, isobutyl acrylate, 2-ethylhexyl acrylate, benzyl methacrylate, phenoxyethyl acrylate, cyclohexyl acrylate, and glycidyl acrylate, styrenics such as styrene, α-methylstyrene, 3- and 4-chloromethylstyrene, halogen-substituted styrenes, and alkyl-substituted styrenes, vinyl halides and vinylidene halides, N-alkylated acrylamides and methacrylamides, vinyl esters such as vinyl acetate and vinyl benzoate, vinyl ether, allyl alcohol and its ethers and esters, and unsaturated ketones and aldehydes such as acrolein and methyl vinyl ketone, isoprene, butadiene and acrylonitrile. Preferably, the monomers will be aliphatic acrylic esters or methacrylic esters.

In addition, small amounts, typically less than 20% of the total weight of the polymerizeable solids, of one or more water-soluble ethylenically unsaturated monomer can be used. These monomers may be ionic or nonionic. Such monomers include but are not necessarily limited to anionic ethylenically unsaturated monomers such as 2-phosphatoethyl acrylate potassium salt, 3-phosphatopropyl methacrylate ammonium salt, acrylamide, methacrylamides, maleic acid and salts thereof, sulfopropyl acrylate and methacrylate, acrylic and methacrylic acids and salts thereof, N-vinylpyrrolidone, acrylic and methacrylic esters of alkylphosphonates, styrenics, acrylic and methacrylic monomers containing amine ammonium functionalities, styrenesulfonic acid and salts thereof, acrylic and methacrylic esters of alkylsulfonates, vinylsulfonic acid and salts thereof. Nonionic monomers may include monomers containing hydrophilic, nonionic units such as poly(ethylene oxide) segments, carbohydrates, amines, amides, alcohols, polyols, nitrogen-containing heterocycles, and oligopeptides. Examples include, but are not limited to poly(ethylene oxide) acrylate and methacrylate esters, vinylpyridines, hydroxyethyl acrylate, glycerol acrylate and methacrylate esters, (meth)acrylamide, and N-vinylpyrrolidone.

The polymer particles of this invention may further comprise monomers containing at least two ethylenically unsaturated chemical functionalities. These functionalities may be vinyl groups, acrylates, methacrylates, vinyl ethers and vinyl esters. Monomers include, but are not limited to aromatic divinyl compounds such as divinylbenzene, divinylnaphthalene or derivatives thereof, diethylene carboxylate esters and amides such as ethylene glycol dimethacrylate, diethylene glycol diacrylate, 1,4 butanediol diacrylate, 1,4 butanediol dimethacrylate, 1,3 butylene glycol diacrylate, 1,3 butylene glycol dimethacrylate, cyclohexane dimethanol diacrylate, cyclohexane dimethanol dimethacrylate, diethylene glycol diacrylate, diethylene glycol dimethacrylate, dipropylene glycol diacrylate, dipropylene glycol dimethacrylate, ethylene glycol diacrylate, ethylene glycol dimethacrylate, 1,6 hexanediol diacrylate, 1,6 hexanediol dimethacrylate, neopentyl glycol diacrylate, neopentyl glycol dimethacrylate, tetraethylene glycol diacrylate, tetraethylene glycol dimethacrylate, triethylene glycol diacrylate, triethylene glycol dimethacrylate, tripropylene glycol diacrylate, tripropylene glycol dimethacrylate, pentaerythritol triacrylate, trimethylolpropane triacrylate, trimethylolpropane trimethacrylate, dipentaerythritol pentaacrylate, di-trimethylolpropane tetraacrylate, pentaerythritol tetraacrylate, divinyl esters such as divinyl adipate, and other divinyl compounds such as divinyl sulfide or divinyl sulfone compounds of allyl methacryl ate, allyl acryl ate, cyclohexanedimethanol divinyl ether diallylphthalate, diallyl maleate, dienes such as butadiene and isoprene and mixtures thereof.

In a preferred embodiment, the polymer particles are rich in specific functionalities which impart upon the particle dispersibility in a desired carrier solvent, compatibility with the microarray's matrix, or the ability to form chemical bonds with biological probe molecules. These chemically active functionalities may be present on the surface of the particle or on stabilizer polymer strands which are covalently grafted, chemisorbed, or physically adsorbed to the particle surface. These chemical functionalities may be, but are not limited to thiols, primary amines, secondary amines, tertiary amines, quaternary ammoniums, phosphines, alcohols, carboxylic acids, primary or secondary amines, vinylsuflonyls, aldehydes, epoxies, hydrazides, succinimidyl esters, carbodiimides, maleimides, iodoacetyls, isocyanates, isothiocyanates, aziridines, sulfonates. Preferably, these functionalities will be carboxylic acids, primary amines, secondary amines, or carboxylic acids.

The polymer particles useful in this invention may be prepared by any method known in the art for preparing particles of 0.1-1 micron in mean diameter. Especially useful methods include emulsion, miniemulsion, and solvent evaporation methods. Such techniques are reviewed in {Arshady, R. “Suspension, Emulsion, and Dispersion Polymerization: a Methodological Survey” Coloid. Polym. Sci. 1992, 270, 717-732 and in Lovell, P. A.; El-Aaser, M. S. “Emulsion Polymerization and Emulsion Polymers”,; Wiley: Chichester, 1997.

Emulsion polymerization is a widely used technique which has been extensively described in literature, both patent and non-patent. Production of synthetic latexes via emulsion polymerization is well-known. Among the polymers commonly produced by emulsion polymerization are styrene-butadiene copolymers, acrylic polymers and copolymers, and polyvinylacetate. Polymers prepared by emulsion polymerization are widely used as binders in water-based latex paints for both interior and exterior use. Emulsion polymerization is also used to prepare polymer foams and polymers used as coatings.

Emulsion polymerization utilizes the following ingredients: water, a monomer or mixture thereof, a surfactant or mixture thereof, and a polymerization initiator. The monomer or mixture thereof is typically dispersed into droplets and polymer particles are formed during the polymerization with the aid of a surfactant or mixture thereof with the aid of an agitator. Monomer droplet diameters are typically from 1 to 10 microns.

Emulsion and miniemulsion polymerizations have many similarities but the particle nucleation and reagent transplant phenomena are very different. Conventional emulsion polymerization starts with a monomer emulsion comprised of relatively large (in the range of 1 to 10 microns) monomer droplets and significant free or micellar emulsifier. Particle nucleation takes place early in the reaction via homogeneous (water phase) reactions or via free radical entry into monomer-swollen micelles. Radicals may enter the monomer droplets but this phenomenon is generally discounted because of the relatively small droplet surface area. Nucleation stops or slows significantly after the surface area of the particles becomes sufficient to adsorb all of the emulsifier. The major locus of polymerization thereafter is in the nucleated particles. The reagents (monomer, chain transfer agents) move from the monomer droplets to the reaction sites in the particles.

Miniemulsion polymerization, by contrast, begins with submicron droplets which are able to accommodate most of the added emulsifier. High intensity fluid deformation and a cosurfactant are employed to generate and stabilize the small droplet size miniemulsion. Particle nucleation is primarily via droplet penetration and, if most droplets are nucleated, the reagents are located at the polymerization sites and mass transport, except for the radicals, is not involved. Either water-soluble or oil-soluble initiators may be employed in miniemulsion polymerization.

Monomer droplet size instability is observed in monomer emulsions. The smaller monomer droplets will disappear by two mechanisms. The first is flocculation into larger droplets. This can be effectively prevented by providing an adequate layer of surfactant at the droplet surface. The second is Ostwald ripening. This phenomenon consists of the diffusion of monomer out of the smaller droplets and into the larger ones. The polymer does not so diffuse. The net effect is a reduction in interfacial surface area, and hence, of surface free energy. In an un polymerized conventional emulsion (which will be called herein a “macroemulsion”), the disappearance of the small droplets takes place in seconds. This precludes the nucleation of these droplets into polymer particles. In a miniemulsion, a combination of high shear and a cosurfactant are used. The high shear generates very small monomer droplets. The cosurfactant retards Ostwald ripening so that the small droplets can resist diffusional instability. The small droplets can then compete effectively for water-borne free radicals, and the locus of nucleation becomes predominantly the monomer droplets. Common cosurfactants include hexadecane, cetyl alcohol, and monomer-soluble polymer.

Polymer particles useful in this invention may be made by solvent evaporation. This involves first forming a solution of a polymer in a solvent that is immiscible with water (along with any addenda), and then suspending the polymer-solvent solution in water containing a surfactant, dispersant, or emulsifier. The resulting suspension is subjected to high shear action to reduce the size of the polymer-solvent droplets. The shearing action is optionally removed and the polymer-solvent droplets coalesce to the extent allowed by the dispersant to form coalesced polymer-solvent droplets. The solvent is removed from the drops to form solidified polymer particles which are then optionally isolated from the suspension by filtration or other suitable means. Any suitable solvent that will dissolve the polymer and which is also immiscible with water may be used.

Another class of fillers useful in this invention includes soluble polymers. These polymers may be linear or branched, natural or synthetic, and will be soluble in water or water-miscible solvents such as water-miscible alcohols (for example, methanol, ethanol, isopropanol, 1-methoxy-2-propanol and n-propanol), methyl ethyl ketone, tetrahydrofuran, acetonitrile and acetone. The polymers may be of any class or type provided that they have some degree of compatibility with gelatin. For purposes of the present invention, the polymer is defined as compatible with gelatin if the dried layer comprising the mixture of the polymer and gelatin does not exhibit any unacceptable physical, optical, or solubility properties that would prevent it's use for the invention. Some degree of microscopic phase separation between the polymer and the gelatin is permitted provided that the filled system is optically clear. For the purposes of this document, we will define “optically clear” as having a test result of less than 0.5% haze using the ASTM D-1003 standard test method for haze and luminous transmittance of transparent plastics. Preferred polymers include polysaccharides such as carboxymethyl cellulose, hydroxyethyl cellulose, agar-agar, arrowroot, guar, dextran, pullulan, carrageenan, tragacanth, xanthan, rhamsan, proteinaceous and polypeptide materials such as albumin and polylysine. Synthetic polymers which are particularly useful include those which contain >1.7 mEq/g of amide, amine, and heterocyclic nitrogen groups. Preferably the polymers will have >3 Meq/g of amide, amine, and heterocyclic nitrogen groups. These polymers include, but are not limited to such as poly (alkyl oxazolines), polyethyleneimine, and addition polymers and copolymers of N-vinylpyrrolidone, vinylamine, diallylamines, N-vinylimidazole, 2 and 4-vinylpyridines, 2-Aminoethyl methacrylate hydrochloride, N-(3-Aminopropyl)methacrylamide hydrochloride, 2-(tert-Butylamino)ethyl methacrylate Diallylamine, 2-(iso-Propylamino)ethylstyrene, 2-(N,N-Diethylamino)ethyl methacrylate, 2-(Diethylamino)ethylstyrene, 2-(N,N-Dimethylamino)ethyl acrylate, N-[2-(N,N-Dimethylamino)ethyl]methacrylamide, 2-(N,N-Dimethylamino)ethyl methacrylate, N-[3-(N,N-Dimethylamino)propyl]acrylamide, N-[3-(N,N-Dimethylamino)propyl]methacrylamide, 4-Vinylpyridine, N-Methylolacrylamide, N— Acryloylmorpholine, N-(3-Aminopropyl)methacrylamide hydrochloride, N-(iso-Butoxymethyl)methacrylamide, N,N-Diallylacrylamide, N,N-Diethylacrylamide N,N-Dimethylacrylamide, N-[2-(N,N-Dimethylamino)ethyl]methacrylamide, N-[3-(N,N-Dimethylamino)propyl]methacrylamide, N,N-Dimethylmethacrylamide, N-Ethylmethacrylamide, N-(2-Hydroxypropyl)methacrylamide, N-Methacryloylmorpholine, N-Methylolacrylamide, N-(Phthalimidomethyl)acrylamide, N-iso-Propylacrylamide, acrylamide, and N-Methylmethacrylamide.

The inorganic particles in the filled layers useful for this invention include any inorganic oxide, including silica, colloidal silica, silicon oxide dispersions such as those available from Nissan Chemical Industries and DuPont Corp., fumed silica, aluminum oxide, colloidal alumina, fumed alumina, calcium carbonate, kaolin, talc, calcium sulfate, natural or synthetic clay, barium sulfate, barium sulfate mixtures with zinc sulfide, inorganic powders such as y-aluminum oxide, chromium oxide, iron oxide, tin oxide, doped tin oxide, alumino-silicate, titanium dioxide, silicon carbide, titanium carbide, and diamond in fine powder, as described in U.S. Pat. No. 5,432,050, titanium dioxide, zinc oxide, or mixtures thereof. Colloidal materials may also be used as fillers.

A dispersing agent, or wetting agent can be present to facilitate the dispersion of the filler particles. This helps to minimize the agglomeration of the inorganic particles. Useful dispersing agents include, but are not limited to, fatty acid amines and commercially available wetting agents such as Solsperse® sold by Zeneca, Inc. (ICI). Preferred inorganic particles are colloidal silica, aluminum oxide, calcium carbonate, and barium sulfate.

The organic particles and inorganic particles can be of any size, however, it is preferable that their mean particle diameter be of less than 1.0 micrometers, preferably from 0.005 to 0.5 micrometers, and most preferably from 0.01 to 0.2 micrometers. The amount of filler particles used should be in an amount insufficient to impart porosity due to interstitial voids to the layers. The filler comprises from 1 to 85% by weight of an individual layer. The particles preferably comprise from 25 to 80% by weight of an individual layer.

In the present invention, a support is coated with at least one layer of filler and gelatin, and at least one functional compound with functional groups capable of interacting with and capable of interacting with a biological capture agent. These functional groups may be independently the same or different from each other.

In a preferred embodiment, the support is coated with a filled gelatin layer and a functional compound comprising at least a functional group A capable of interacting with gelatin and a functional group B capable of interacting with a biological capture agent. The groups A and B may be the same or different and are connected by a linking group L capable of interacting with A and B. In a preferred embodiment, the functional compound is a trifunctional compound attached to or dispersed in the gelatin. In general, the trifunctional molecule is represented as A-L-B, in which A and B are chemical functionalities that are capable of reacting or interacting with the gelatin and biological capture agent molecules to be immobilized on the substrate and L is a linkage group connecting group A to group B. Preferably, L is a di-radical of such a length that the shortest through-bond path between the ends that connect A to B is not greater than 10 atoms. The reaction or interaction between the functional compound and the gelatin and the biological capture agent may preferably be a physical binding or a chemical reaction.

There are two classes of trifunctional agents: 1). homofunctional agents, where the A and B groups are identical, and 2). heterofunctional agents, wherein the A and B groups are different. Commonly used A and B groups may include aldehyde, epoxy, hydrazide, vinyl sulfone, succinimidyl ester, carbodiimide, maleimide, dithio, iodoacetyl, isocyanate, isothiocyanate, aziridine.

The linking group L comprises any reasonable combination of relatively non-labile covalently bonded chemical units sufficient to connect the two functionalities A and B. These chemical units may consists of, but are not necessarily limited to, a single bond, a carbon atom, an oxygen atom, a sulfur atom, a carbonyl group 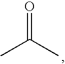
a carboxylic ester group 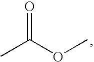
a carboxylic amide group 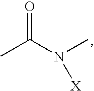
a sulfonyl group 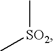
a sulfonamide group 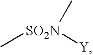
an ethyleneoxy group, a polyethyleneoxy group, or an amino group 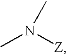
where substituents X, Y, and Z are each independently a hydrogen atom, or an alkyl group of 1-10 carbon atoms, and linear or branched, saturated or unsaturated alkyl group of 1 to 10 carbon atoms (such as methyl, ethyl, n-propyl, isopropyl, t-butyl, hexyl, decyl, benzyl, methoxymethyl, hydroxyethyl, iso-butyl, and n-butyl), a substituted or unsubstituted aryl group of 6 to 14 carbon atoms (such as phenyl, naphthyl, anthryl, tolyl, xylyl, 3-methoxyphenyl, 4-chlorophenyl, 4-carbomethoxyphenyl and 4-cyanophenyl), and a substituted or unsubstituted cycloalkyl group of 5 to 14 carbon atoms such as cyclopentyl, cyclohexyl, and cyclooctyl), a substituted or unsubstituted, saturated or unsaturated heterocyclic group (such as pyridyl, primidyl, morpholino, and furanyl), a cyano group. Some solubilizing groups may also be introduced into A-L-B and examples of these solubilizing groups include, but are not limited to, carboxylic acid, sulfonic acid, phosphonic acid, hydroxamic acid, sulfonamide, and hydroxy groups (and their corresponding salts). A and B may also be in the form of readily reactive functionalities towards crosslinkers, examples include but not limited to carboxy, amino, and chloromethyl. A and B may be affinity tags that are capable of interacting non-covalently with the biological capture agents intended to be immobilized onto the substrate. For example, some commonly used tag systems include, but are not limited to, streptavidin and biotin, histidine tags and nickel metal ions, glutathione-S-transferase and glutathione. One skilled in the art should be able to create a fusion biological capture agent using recombination DNA technology and an element of tag recognition unit may be introduced into biological capture agent in this way.

The trifunctional compound A-L-B comprises a compound rich in chemical functionalities that will immobilize biological compounds. Such functionalities may include aldehyde, epoxy, hydrazide, vinyl sulfone, succinimidyl ester, carbodiimide, maleimide, dithio, iodoacetyl, isocyanate, isothiocyanate, and aziridine.

In another preferred embodiment, the functional compound comprises a polymer, which may be represented by the structure in Formula I, and consist of the polymerization products of a “G” monomer, which affords a polymer with favorable solubility properties, and an “H” monomer.
-[G]ₓ-[H]_{y}—
wherein

G is a polymerized α,β-ethylenically unsaturated addition polymerizeable monomer which imparts water-solubility to the polymer. The monomer from which G may be derived include both ionic and nonionic monomers. Ionic monomers may include, for example, anionic ethylenically unsaturated monomers such as 2-phosphatoethyl acrylate potassium salt, 3-phosphatopropyl methacrylate ammonium salt, acrylamide, methacrylamides, maleic acid and salts thereof, sulfopropyl acrylate and methacrylate, acrylic and methacrylic acids and salts thereof, N-vinylpyrrolidone, acrylic and methacrylic esters of alkylphosphonates, styrenics, acrylic and methacrylic monomers containing amine ammonium functionalities, styrenesulfonic acid and salts thereof, acrylic and methacrylic esters of alkylsulfonates, vinylsulfonic acid and salts thereof. Nonionic monomers may include monomers containing hydrophilic, nonionic units such as poly(ethylene oxide) segments, carbohydrates, amines, amides, alcohols, polyols, nitrogen-containing heterocycles, and oligopeptides. Examples include, but are not limited to poly(ethylene oxide) acrylate and methacrylate esters, vinylpyridines, hydroxyethyl acrylate, glycerol acrylate and methacrylate esters, (meth)acrylamide, and N-vinylpyrrolidone. Preferably, G is the polymerized form of acrylamide, sodium 2-acrylamido-2-methanepropionate, sulfopropyl acrylate and methacrylate salts, or sodium styrenesulfonate.

Monomer H is the polymerized form of a vinylsulfone or vinylsulfone precursor unit covalently bound to a polymerizeable α,β-ethylenically unsaturated function by an organic spacer which consists of Q and L, of which Q is an optional component.

More than one type each of G and H monomers may be present in the same polymer.

Formula II represents a preferred polymer for forming the functional polymer: 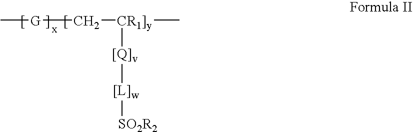
wherein R₁ is a hydrogen atom or a C₁-C₆ alkyl group. Preferably R1 is a hydrogen atom.
Q is —CO₂—, or CONR₁.;
v is 1 or 0;
w is 1-3;
L is a divalent linking group containing at least one linkage selected from the group consisting of —CO₂— and —CONR₁, and containing 3-15 carbon atoms, or a divalent atom containing at least one linkage selected from the group consisting of —O—, —N(R₁)—, —CO—, —SO—, —SO₂—, —SO₃—, —SO₂N(R₁)—, —N(R₁)CON(R₁)—and —N(R₁)CO₂—, and containing 1-12 carbon atoms in which R₁ has the same meaning as defined above;
R₂ is —CH═CH2 or —CH2-CH2X₁ wherein X₁ is a substituent replaceable by a nucleophilic group or releasable in the form of HX₁ by a base. X₁ may be, but is not necessarily limited to —S₂O₃⁻, —SO₄—, —Cl, —Br, —I, quaternary ammonium, pyridinium, and —CN, and sulfonate esters (such as mesylate and tosylate);
x and y both represent molar percentages ranging from 10 to 90 and 90 to 10. Preferably, x and y range from 25 to 75 and 75 to 25, respectively.

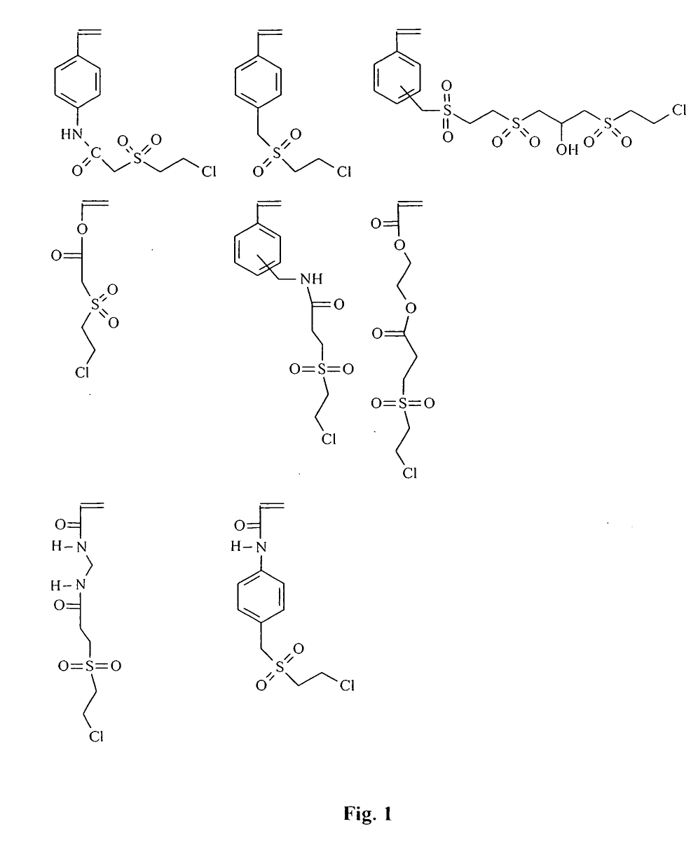

Polymers preferred for this embodiment consist of the polymerization products of a “G” monomer, and an “H” monomer, which contains the vinylsulfone moiety or, more preferably, a vinylsulfone precursor function, such as a sulfonylethyl group with a leaving group in the β-position. In a preferred embodiment of this invention, a polymer containing pendant vinylsulfone or vinylsulfone precursor units may be reacted with the gelatin in order to provide a polymer scaffold. Vinylsulfone and vinylsulfone-containing precursor “H” monomers useful in this embodiment include, but are not necessarily limited to those compounds disclosed in U.S. Pat. Nos. 4,548,869 and 4,161,407 (incorporated herein by reference) as well as those compounds in FIG. 1.

Additional monomers may be incorporated in order to modify properties such as glass transition temperature, surface properties, and compatibility with other formulation components as needed for specific applications. Selection of additional monomers will be application dependent and will be obvious to one skilled in the art.

Although the polymer may have any molecular weight, molecular weights (Mn) from 1000 to 200,000 AMU are preferred. Molecular weights from 2000 to 50,000 AMU are especially preferred provided that the polymer is soluble in water or a mixture of water and water-miscible solvents (such as methanol, ethanol, acetone, or tetrahydrofuran).

The present invention may attain very high densities of chemical moieties that are useful in the immobilization of proteins and biological compounds. To accomplish this, the present invention employs either a “polymer scaffold” strategy for attaching the biological capture agent to the filled gelatin layer or attachment of the biological capture agent to the functional compound dispersed in the filled gelatin layer.

The gelatin used in the invention may be chemically modified with the functional compound either before, during or after the coating process to create more chemical functionalities that can react or interact with biologically active molecules or assemblies intended to be attached on this substrate. In general, there are two ways to prepare a reactive surface for biological capture agent immobilization using a gelatin coating method. In the first approach, the chemical agent or polymer scaffold may be mixed with gelatin and filler and coated on a solid support. In the second approach, a filled gelatin coating is prepared on a solid support, and a coating containing chemical agents, for example, A-L-B, polymer scaffold, to affix the reactive chemistry to the gelatin surface is applied to the filled gelatin layer.

One strategy is referred to herein as a “polymer scaffold” strategy. For the purposes of this invention, the term “polymer scaffold” refers to a linear or branched polymer, rich in specific functionalities, that extends out in a 3-dimensional fashion from a surface. In this case, functional groups consist of chemical units capable of immobilizing biological capture agents. The polymer scaffold may be prepared either by the application of a precursor polymer, rich in units that are capable of being converted into chemical functions that will immobilize biological capture agents, to the gelatin surface and conversion to a biological-receptive form by post-treatment with a chemical agent or by direct application of the biologically receptive form to the gelatin surface.

In a preferred embodiment, the precursor polymer will attach to the functional compound in or on the filled gelatin layer. The precursor polymer may be rich in such reactive units as thiols, amines, phosphines, alcohols, or carboxylic acids. Preferably the reactive unit is a primary or secondary amine. Specific precursor polymers which may be used for this purpose may include poly (propyleneimine) and polymers and copolymers of N-aminopropyl (meth)acrylamide and secondary amine derivatives thereof, N-aminoethyl (meth)acrylate and secondary amine forms thereof, diallyamine, vinylbenzylamine, vinylamine, (meth)acrylic acid, vinylbenzyl mercaptan, and hydroxyethyl(meth)acrylate. Preferably, the polymer is poly(vinylamine), poly(propyleneimine), or poly(N-aminopropyl methacrylamide).

The direct affixing of the scaffold polymer to the surface of the gelatin may also be achieved using any chemical agent or technique that is known to result in the formation of a covalent bond between the reactive units of the polymer and either the amine or carboxylic acid functionality of the gelatin. By “affixed” it is meant that the precursor polymer is applied to the gelatin surface and adheres to the gelatin by any of a number of chemical and physical attractive mechanisms including ionic interactions, covalent bonds, coordinative bonds, hydrogen bonds, and Van-der-Waals interactions. For example, a dehydrating agent such as a carbodiimide, a pyridyl dication ether, or a carbamoylpyridinium compound may be used to bind an amine-containing polymer or a carboxylic acid-containing polymer to a gelatin surface via amide bonds. Similarly, a bis(vinylsulfonyl) compound may be used to bind poly(ethyleneimine) to a gelatin surface. Once the scaffold polymer is affixed to the gelatin surface, it is then treated with an excess of the appropriate compound to afford the reactive surface with a high level of reactive units.

In one embodiment, the polymer scaffold may be the functional compound. Additionally, more than one type of polymer may be affixed to the same gelatin substrate.

The support utilized in the present invention may be modified prior to coating the layer comprising filler and gelatin and the functional compound. In a preferred embodiment of this invention, a support surface is coated with an interlayer or subbing layer to provide a hydrophilic surface for the subsequent coating of filled gelatin layer.

Coating a hydrophilic binder, for example, gelatin, onto glass is a very demanding task. A compatible interlayer or subbing layer is frequently applied between glass and the binder. Such adhesive interlayer desirably have the following properties: 1) it is a thin film that does not have any optical interferences for the biological microarray applications; 2) it does not contain any components that may have chemical interference to the biological capture agent attachment chemistry incorporated into the binder or onto binder surface; and 3) it can be readily manufactured.

When gelatin is coated on a solid support, for example, glass, plastic, or metal, an interlayer is desirable to prevent frilling of the gelatin coating when the filled gelatin coating is wet during any biological processing, or stripping, when the filled gelatin coating is dry. Generally, an interlayer consists of a film forming hydrophilic colloidal material or hydrophilic binder. In addition to providing adequate adhesive force for binding the filled gelatin layer, the interlayer is preferably optically transparent and not fluorescent.

This interlayer may be an adhesive layer such as, for example, halogenated phenols, partially hydrolyzed vinyl chloride-co-vinyl acetate polymer, vinylidene chloride-methyl acrylate-itaconic acid terpolymer, a vinylidene chloride-acrylonitrile-acrylic acid terpolymer, or a glycidyl (meth)acrylate polymer or copolymer. Other chemical adhesives, such as polymers, copolymers, reactive polymers or copolymers, that exhibit good bonding between the layer and the support can be used.

The interlayer may also be a subbing layer. The polymeric binder in a subbing layer employed in the invention is preferably a water soluble or water dispersible polymer such as poly(vinyl alcohol), poly(vinyl pyrrolidone), proteins, protein derivatives, gelatin, for example, alkali-treated gelatin such as cattle bone or hide gelatin, or acid treated gelatin such as pigskin gelatin, and gelatin derivatives, for example, acetylated gelatin, and phthalated gelatin, a cellulose ether, a poly(oxazoline), a poly(vinylacetamide), partially hydrolyzed poly(vinyl acetate/vinyl alcohol), poly(acrylic acid), poly(acrylamide), poly(alkylene oxide), a sulfonated or phosphated polyester or polystyrene, zein, chitin, chitosan, dextran, pectin, a collagen derivative, collodian, agar-agar, arrowroot, guar, carrageenan, tragacanth, xanthan, rhamsan; a latex such as poly(styrene-co-butadiene), a polyurethane latex, a polyester latex, or a poly(acrylate), poly(methacrylate), poly(acrylamide) or copolymers thereof.

In the case of gelatin as the preferred interlayer material, an organic solvent, or a mixture of solvents, should also be included in the formulation. Examples of such organic solvent include, but not limited to, acetone, alcohol, ethyl acetate, methylene chloride, ether, or a mixture of the foregoing.

In order to uniformly mix gelatin with these organic solvent, a dispersing aid may be added to the formulation, for example, organic acids or bases. To improve adhesive strength of the interlayer, silicate salt, for example, sodium silicate, is also included in the interlayer formulation. To improve the physical strength of the interlayer, it is preferred that gelatin in the interlayer is hardened using one or more crosslinking agents. Examples of gelatin hardening agents can be found in standard references such as The Theory of the Photographic Process, T. H. James, Macmillan Publishing Co., Inc. (New York 1977) or in Research Disclosure, September 1996, Number 389, Part IIB (hardeners). Inorganic hardening agents are preferred over organic hardening agents.

It has been recognized in the art, as described in U.S. Pat. No. 3,864,132, and British Pat. No. 1,066,944, that a hydrophilic colloid layer can be firmly bonded to a hydrophobic polymer supporting surface by means of an inorganic oxide adhesive layer which is contiguous to the supporting surface and to the hydrophilic colloid layer. Such adhesive layers, commonly referred to as subbing layers in the arts, are binderless layers which consist essentially of inorganic metal oxide and are capable of bonding directly and tenaciously to both hydrophilic colloid layers and to hydrophobic polymeric support surfaces to perform the function heretofore performed by considerably more complex polymer layers. The term “binderless layer” refers to a layer that is substantially free of organic adhesive materials and refers particularly to the absence of those organic adhesive and binder materials commonly utilized in the arts, such as natural and synthetic polymeric binders and colloidal vehicles. The binderless adhesive layer may be formed of crystalline or amorphous inorganic oxides. Oxides of silicon, such as silicon monoxide and silicon dioxide, are preferred inorganic oxides, since they are substantially water insoluble and chemically inert in photographic processing and use environments and are essentially transparent. Silicon oxides are also preferred since they may be vapor deposited by heating to vaporization temperatures that are low as compared to those utilized for vaporizing the other inorganic oxides utilized in the practice of this invention. Aluminum oxide, boron-silicon oxide, magnesium oxide, tantalum oxide and titanium oxide as well as mixtures thereof are particularly suited to the practice of this invention. The inorganic oxide adhesive layer may be utilized on glass support.

When a polymer support is used, a surface treatment is desirable to render the appropriate adhesiveness for binding the filled gelatin layer. Useful surface treatments may include chemical treatments, such as strong acids, based, oxidants, and reductants and physical treatments such as corona discharge treatment, flame treatment, ultraviolet ray treatment, high frequency treatment, active plasma treatment, laser treatment, glow discharge, LTV exposure, or electron beam treatment, as described in U.S. Pat. Nos. 2,764,520, 3,497,407, 3,145,242, 3,376,208, 3,072,483, 3,475,193, 3,360,448, and British Pat. No. 788,365.

Polymer supports may be surface-treated with adhesion-promoting agents including dichloroacetic acid and trichloroacetic acid, phenol derivatives such as resorcinol and p-chloro-m-cresol. Polymer supports may be solvent washed prior to overcoating with a subbing interlayer, for example, a gelatin interlayer. In addition to surface treatment or treatment with adhesion promoting agents, additional adhesion promoting primer or tie layers containing polymers such as vinylidene chloride-containing copolymers, butadiene-based copolymers, glycidyl acrylate or methacrylate-containing copolymers, maleic anhydride-containing copolymers, condensation polymers such as polyesters, polyamides, polyurethanes, polycarbonates, mixtures and blends thereof may be applied to the polyester support. Particularly preferred primer or tie layers comprise a chlorine containing latex or solvent coatable chlorine containing polymeric layer. Vinyl chloride and vinylidene chloride containing polymers are preferred as primer or subbing layers.

An adhesive interlayer as described in U.S. Pat. Nos. 3,511,661, and 3,860,426, may be used on metal support. For instance, aluminum is a preferred metal support in lithographic plate industry due to its availability and low cost. Generally an anodic oxidation as described in U.S. Pat. Nos. 4,608,131, 4,092,169, and 4,446,221, is carried out on aluminum support surface before the application of the adhesive interlayer.

The total thickness of the combined layers may range from 0.1 to 10 μm, preferably from 0.4 to 5 μm. Each layer may have a different thickness relative to the other layers. The coating thickness is determined through the need for a particular application. For example, this invention can be used for a printed antibody array, or for an affinity capture surface for matrix assisted laser desorption mass spectrometry. One skilled in the art should be able to determine the appropriate thickness relative to the intended use.

The filled layers or interlayers may include additives. Lubricating agents may be one type of additive. Lubricants and waxes useful for the invention include, but are not limited to, polyethylenes, silicone waxes, natural waxes such as carnauba, polytetrafluoroethylene, fluorinated ethylene propylene, silicone oils such as polydimethylsiloxane, fluorinated silicones, functionalized silicones, stearates, polyvinylstearate, fatty acid salts, and perfluoroethers. Aqueous or non-aqueous dispersions of submicron size wax particles such as those offered commercially as, but not limited to, dispersions of polyolefins, polypropylene, polyethylene, high density polyethylene, microcrystalline wax, paraffin, natural waxes such as carnauba wax, and synthetic waxes from such companies as Chemical Corporation of America (Chemcor), Inc., Michelman Inc., Shamrock Technologies Inc., and Daniel Products Company, are useful.

In order to obtain adequate coatability, additives known to those familiar with such art such as surfactants, coating aids, defoamers, and alcohol may be used. Coating aids and surfactants include, but are not limited to, nonionic fluorinated alkyl esters such as FC-430®, FC-431 (, FC-10®, FC-171® sold by Minnesota Mining and Manufacturing Co., Zonyl® fluorochemicals such as Zonyl-FSN®, Zonyl-FTS®, Zonyl-TBS®, Zonyl-BA® sold by DuPont Corp. and FT-248 (sold by Bayer), other fluorinated polymer or copolymers such as Modiper F600® sold by NOF Corporation, polysiloxanes such as Dow Corning DC 1248®, DC200®, DC510®, DC 190® and BYK 320®, BYK 322®, sold by BYK Chemie and SF 1079®, SF1023®, SF 1054®, and SF 1080® sold by General Electric, and the Silwet® polymers sold by Union Carbide, polyoxyethylene-lauryl ether surfactants, sorbitan laurate, palmitate and stearates such as Span® surfactants sold by Aldrich, poly(oxyethylene-co-oxypropylene) surfactants such as the Pluronic® family sold by BASF, and other polyoxyethylene-containing surfactants such as the Triton X® family sold by Union Carbide, ionic surfactants, such as the Alkanol® series, such as Alkanol XC, sold by DuPont Corp., and the Dowfax® family sold by Dow Chemical. Specific examples are described in MCCUTCHEON's Volume 1: Emulsifiers and Detergents, 1995, North American Edition.

In general, the biological microarray of the present invention may be prepared by depositing a filled gelatin layer combined with a functional compound onto a solid support, followed by attachment of biological capture agents at pre-defined locations. The functional compound may be introduced either during or after the gelatin coating onto a solid support. The support may optionally be modified prior to application of the coated layers. In one preferred embodiment, the functional compound is disposed in the layer comprising filler and gelatin. In another preferred embodiment, the functional compound is disposed on the layer comprising filler and gelatin.

The layers described above may be coated by conventional coating means onto a support material commonly used in this art. Coating methods may include, but are not limited to, wound wire rod coating, knife coating, slot coating, slide hopper coating, gravure coating, spin coating, dip coating, skim-pan-air-knife coating, multilayer slide bead, doctor blade coating, gravure coating, reverse-roll coating, curtain coating, and multilayer curtain coating. Some of these methods allow for simultaneous coatings of more than one layer, which is preferred from a manufacturing economic perspective if more than one layer or type of layer needs to be applied. Coating methods are broadly described by Edward Cohen and Edgar B. Gutoff in Chapter 1 of “Modern Coating And Drying Technology”, (Interfacial Engineering Series, v.1), (1992), VCH Publishers Inc., New York, N.Y. Known coating and drying methods are described in further detail in Research Disclosure no.308119, published December 1989, pages 1007 to 1008. Slide coating is preferred, in which several layers may be simultaneously applied. However, sequential coating of the several layers may also be utilized. The support may be stationary, or may be moving so that the coated layer is immediately drawn into drying chambers. In general, a fluid coating composition contains a binder, a solvent to dissolve or suspend the components, and optional additives such as surfactants, dispersants, plasticizers, biocides, cross-linking agents for toughness and insolubility, and conductive materials to minimize static buildup. All the components are mixed and dissolved or dispersed, and the coating fluid is sent to an applicator where it is applied to a substrate by one of several coating techniques. Heat is then applied to the coating to evaporate the solvent and produce the desired film, or the coating is solidified by the action of ultraviolet radiation or an electron beam.

It may be desirable to apply interlayers to the solid support using an in-line process during the microarray substrate manufacture. However, it may also be applied in separate processes. To achieve ultra thin film coating with the interlayer application, it is desirable that the interlayer is coated using either gravure method, as described in U.S. Pat. Nos. 3,283,712, 3,468,700, and 4,325,995, or wicked coating method, as described in 3,000,349, 3,786,736, 3,831,553, and 4,033,290.

The most suitable coating method-including the coating speed-will depend on the quality and functionality desired and the materials being used, for example, the substrate, the solvent, or weight and viscosity of the coating. For a single layer format, suitable coating methods may include dip coating, rod coating, knife coating, blade coating, air knife coating, gravure coating, forward and reverse roll coating, and slot and extrusion coating.

Coating speed may also be a determinant in the choice of coating method. Although most methods may be used at low speeds, and all methods have a limiting upper speed, some work better at higher speeds. Curtain coating utilizes a minimum flow to maintain the integrity of the curtain. Therefore, this method is limited to higher speeds if a thin coating is to be obtained. In slide coating of multiple layers, interfacial instabilities are more likely to occur on the slide when the layers are very thin. Higher speeds, with their higher flows and thicker layers on the slide, tend to avoid these instabilities. See, p. 12, “Modern Coating and Drying Technology”, supra.

Once a microarray substrate is modified with the functional compound, biological capture agents may be placed onto the substrate to generate biological microarray content. The biological capture agents may be deposited onto the membrane by physical adsorption. In one embodiment, the biological capture agents may be immobilized onto a substrate through chemical covalent bond.

A protein molecule consists of 20 amino acids that are connected in linear manner covalently. Some proteins may be further modified at selected amino acids through posttranslational processes that include phosphorylation and glycosylation. A protein molecule may be used as a biological capture agent. Biological capture agents are molecules that can interact with biological compounds in high affinity and high specificity. Typically it is desirable to have an affinity binding constant between a biological capture agent and target biological compound greater than 10⁶ M⁻¹. There are several classes of molecules that can be used as biological capture agents on a biological microarray. Antibodies are a class of naturally occurring biological molecules that are capable of binding targets with high affinity and specificity. The properties and protocols of using antibody can be found in “Using Antibodies; A Laboratory Manual”, (Cold Spring Harbor Laboratory Press, by Ed Harlow and David Lane, Cold Spring Harbor, N.Y. 1999). Antigens may also be used as biological capture agents if antibodies are intended targets for detection. Biological scaffolds such as whole protein/enzyme or their fragments may be used as biological capture agents as well. Examples include phosphotases, kinases, proteases, oxidases, hydrolyases, cytokines, or synthetic peptides. Nucleic acid ligands may be used as biological capture agent molecules after in vitro selection and enrichment for their binding affinity and specificity to certain targets. The principle of such selection process can be found in Science, Vol. 249, 505-510, 1990 and Nature, Vol. 346, 818-822, 1990. U.S. Pat. No. 5,110,833 discloses an alternative class of synthetic molecules that can mimic antibody binding affinity and specificity and can be readily prepared by the so called Molecular Imprinting Polymer (MIP). This technology has been reviewed in Chem. Rev. Vol. 100, 2495-2504, (2000).

In practice, a biological microarray is brought into contact with a biological fluid sample. Biological compounds in the sample will adsorb to both areas spotted with specific biological capture agents and areas without biological capture agents. Since the biological microarray is intended to be used for the measurement of specific interactions between biological capture agents on the microarray with certain proteins or other biological molecules in the biological fluid sample, the non-specific binding of sample proteins to non-spotted area would give rise to high background noise. The term non-specific binding refers to the tendency of protein molecules to adhere to a solid surface in a non-selective manner.

This high background noise resulting from the non-specific binding will interfere with reporter signals to be detected from the spotted area unless the non-specific binding is blocked in an appropriate manner. Typically, the biological microarray will be immersed in a solution containing a blocking agent to block the non-specific binding sites before its contact with the intended analyte solution. A commonly used method for blocking protein non-specific binding is to treat the surface of the substrate with a large excess of bovine serum albumin. The non-spotted surface area may also be chemically modified with polyethylene glycol (PEG), phospholipid, or poly lysine to prevent non-specific binding.

The invention can be better appreciated by reference to the following specific embodiments.

### EXAMPLES

### Example 1

Example 1 illustrates possible preparations of organic fillers.

Sample P-1: Synthesis of Dispersion of Organic Filler P-1 (Methyl Methacylate Latex)

Methyl methacrylate (100.00 g, passed over basic alumina) was combined with deionized water (900.00 g), anionic surfactant Aerosol OT-75 (5.33 g, obtained from Cytec as a 75% solution) and sodium bicarbonate (0.50 g) in a 2 liter 3-neck round bottom flask outfitted with a condenser, mechanical stirrer, and nitrogen inlet. The contents were bubble degassed with nitrogen for 10 minutes and the flask was placed in a thermostatted water bath at 60° C. Sodium persulfate (1.00 g) and sodium metabisulfite (0.10 g) were added and the reaction was allowed to stir for 16 hours. 989.24 g of a coagulum-free latex was obtained. The final dispersion concentration was 10.0 wt. % solid particles. The mean particle diameter was found to be 0.0655 microns via photon correlation spectroscopy using a Microtrac UPA150 instrument.

Sample P-2: Synthesis of Dispersion of Organic Filler P-2 (Latex of Butyl Acrylate-co-2-Acrylamido-2-Methylpropane Sulfonic Acid-co-Acetoacetoxyethyl Methacrylate (93:3:4)

To a 2 L 3-neck round bottom flask (“reactor”) outfitted with a mechanical stirrer, reflux condenser, nitrogen inlet, and rubber septum was added 425 mL of demineralized water and 5.0 g of nonionic surfactant 10G (Olin Corp.) were added and the flask was placed in a thermostatted oil bath at 85° C. To a custom blown “header” flask (a standard 3-neck round bottom flask modified with a male Luer-Loc adapter on the bottom) outfitted with a reflux condenser, mechanical stirrer and nitrogen inlet was added 120 g of demineralized water, 1.6 g of sodium hydroxide, 8.52 g of 2-acrylamido-2-methylpropane sulfonic acid solution (Lubrizol®, 50% solution in water), 5.0 g of Olin Surfactant 10G, 5.72 g of acetoacetoxyethyl methacrylate, 129.6 g butyl acrylate, and 0.58 g of potassium persulfate. The header flask was allowed to stir at a rate sufficient to emulsify the monomer mixture. The header and reactor contents were bubble degassed with nitrogen for 20 minutes. 0.384 g of sodium metabisulfite, and 0.576 g of potassium persulfate were added to the “reactor” flask and the contents of the “header” were pumped into the reactor over 45 minutes. The monomer emulsion delivery system consisted of plastic tubing leading from the header flask's luer loc adapter through solvent pump into the ‘reactor flask via the rubber septum. The reactor contents were stirred for four hours at 85° C. The latex was then cooled to 25° C., and 0.2 g of Ottasept (4-Chloro-3-xylenol, a preservative) was added and the latex was filtered through gauze. The total yield of latex was 720 g at 30% solids. The final dispersion concentration was 20.3 wt. % solid particles. The mean particle diameter was found to be 0.0655 microns via photon correlation spectroscopy using a Microtrac UPA 150 instrument.

Sample P-3: Synthesis of Dispersion of Organic Filler P-3 (Latex of Glycidyl Acrylate-co-2-Butyl Acrylate (85:15).

This reaction was carried out using the same apparatus and procedure as that described for Sample P-2, except that a 1 L flask was used as the reactor flask. To the reactor flask was added 200 mL of demineralized water and 9.3 g of Rhodacal® A246/L (a sodium C14-16 olefin sulfonate surfactant available from Rhodia). To the header flask was added 190 ml of demineralized water, 160 g of glycidyl methacrylate, 28 g butyl acrylate, and 9.3 g Rhodacal A246/L. The header flask was allowed to stir at a rate sufficient to emulsify the monomer mixture. The header and reactor contents were bubble degassed with nitrogen for 20 minutes. The Reactor contents were brought to a temperature of 60° C. using a thermostatted water bath, at which point 1.86 g of azobis 4-cyanovaleric acid was added and the contents of the “header” were pumped into the reactor over 4 hours. When the charge was complete the product was cooked for two hours at 60° C. A solution of 0.80 g erythorbic acid in 10 g water was added and a solution of 0.2 g of (35%) hydrogen peroxide in 34 mL of demineralized water was pumped in over 30 minutes. When the charge was complete the product was cooked for one hour at 60° C. The latex was then cooled to 25° C. The product was filtered through a 30 micron cartridge. The total yield of latex was 680 g at 30.8% solids. The mean particle diameter was found to be 0.0340 microns via photon correlation spectroscopy using a Microtrac UPA150 instrument.

### Example 2

Example 2 illustrates the preparation of the filled gelatin based substrate where the filler comprises inorganic and organic filler particles.

Coatings with Inorganic Filler

Preparation of Element 1

A coating composition was prepared from 58.0 wt. % of Snowtex C (a 20 wt. % aqueous colloidal dispersion of silica from Nissan Chemical Industries, Ltd.), 3.0 wt. % gelatin (APO Code 4 gelatin), 13.0 wt. % of a 1.8 wt. % aqueous solution of bisvinylsulfonylmethane (BVSM), 3.0 wt. % of a 10.0 wt. % aqueous solution of anionic surfactant Alkanol-XC (purchased from DuPont), 0.3 wt. % of a 9.0 wt. % aqueous solution of coating aid FT-248 (a fluouro-surfactant purchased from Bayer), and 22.7 wt. % water. The relative proportions of inorganic filler particles to gelatin are therefore 80/20 by weight. The solution was coated onto a support comprised of a glass microscope slide that was previously coated with a poly-L-Lysine subbing layer (purchased from LabScientific, Inc.). The thickness of the dry layer was measured to be about 1.5±0.2 μm.

Preparation of Element 2

A coating composition was prepared, coated, and dried the same as Element 1 except that inorganic filler was Sachtoperse HU-N (a 40 wt. % aqueous colloidal dispersion of barium sulfate from Sachtleben Chemie GmbH) and was 19.0 wt. %, gelatin was 7.5 wt. %, the BVSM solution was 34 wt. %, the FT-248 solution was 0.4 wt. % and the water was 36.1 wt. %. The relative proportions of inorganic filler particles to gelatin are therefore 50/50 by weight. The thickness of the dry layer was measured to be about 1.4±0.2 μm.

Preparation of Element 3

A coating composition was prepared, coated, and dried the same as Element 2 except that the Sachtoperse HU-N was 39.0 wt. %, gelatin was 4.0 wt. %, the BVSM solution was 18.0 wt. %, the Alkanol-XC solution was 4.0 wt. %, and the water was 34.6 wt. %. The relative proportions of inorganic filler particles to gelatin are therefore 80/20 by weight. The thickness of the dry layer was measured to be about 1.1±0.2 μm

Preparation of Element 4

A coating composition was prepared, coated, and dried the same as Element 2 except that the inorganic filler was Nalco® 2329 (a 40 wt. % aqueous colloidal dispersion of silica from Nalco Chemical Company). The relative proportions of inorganic filler particles to gelatin are therefore 50/50 by weight. The thickness of the dry layer was measured to be about 2.0±0.2 μm.

Preparation of Element 5

A coating composition was prepared, coated, and dried the same as Element 3 except that the inorganic filler was Nalco® 2329. The relative proportions of inorganic filler particles to gelatin are therefore 80/20 by weight. The thickness of the dry layer was measured to be about 1.8±0.2 μm.

Preparation of Control Element 6

A coating composition was prepared, coated, and dried the same as Element 1 except that no inorganic filler was added. The gelatin was 11.25 wt. %, the BVSM solution was 51 wt. %, the Alkanol-XC solution was 2.0 wt. %, the FT-248 solution was 0.3 wt. % and the water was 35.45 wt. %. The thickness of the dry layer was measured to be about 1.8±0.2 am.

Coatings with Organic Filler

Preparation of Element 7

A coating composition was prepared, coated, and dried the same as Element 1, except that the filler was dispersion P-1 and was 52 wt. %, the gelatin was 5.2 wt. %, the BVSM solution was 23.0 wt. %, the Alkanol-XC solution was 2.0 wt. %, the FT-248 solution was 0.2 wt. %, and the water was 17.6 wt. %. The relative proportions of latex filler particles to gelatin are therefore 50/50 by weight. The thickness of the dry layer was measured to be about 1.8±0.1 μm.

Preparation of Element 8

A coating composition was prepared, coated, and dried the same as Element 7 except that dispersion P-1 was 81.0 wt. %, gelatin was 2.0 wt. %, the BVSM solution was 9.0 wt. %, and water was 5.8 wt. %. The relative proportions of latex filler particles to gelatin are therefore 80/20 by weight. The thickness of the dry layer was measured to be about 0.6±0.1 μm.

Preparation of Element 9

A coating composition was prepared, coated, and dried the same as Element 7 except that dispersion P-3 was used in place of dispersion P-1 and was 50.0 wt. %, gelatin was 4.0 wt. %, the BVSM solution was 18.0 wt. %, the Alkanol-XC solution was 4.0 wt. %, the FT-248 solution was 0.4 wt. %, and the water was 23.6 wt. %. The relative proportions of latex filler particles to gelatin are therefore 80/20 by weight. The thickness of the dry layer was measured to be about 3.2±0.1 mm.

Preparation of Element 10

A coating composition was prepared, coated, and dried the same as Element 7 except that dispersion P-2 was used in place of dispersion P-1 and was 35.0 wt. %, gelatin was 7.0 wt. %, the BVSM solution was 32.0 wt. %, the Alkanol-XC solution was 3.0 wt. %, the FT-248 solution was 0.3 wt. %, and water was 22.7 wt. %. The relative proportions of latex filler particles to gelatin are therefore 50/50 by weight. The solution was coated and dried the same as Element 1. The thickness of the dry layer was measured to be about 1.7±0.1 μm.

Preparation of Element 11

A coating composition was prepared, coated, and dried the same as Element 3 except that dispersion P-2 was 64.0 wt. %, gelatin was 3.2 wt. %, the BVSM solution was 15.0 wt. %, and water was 14.5 wt. %. The relative proportions of latex filler particles to gelatin are therefore 80/20 by weight. The solution was coated and dried the same as Element 1. The thickness of the dry layer was measured to 1.8±0.1 μm.

Preparation of Control Element 12

A coating composition was prepared, coated and dried the same as Element 9 except that no filler was included. The gelatin was 11.25 wt. %, the BVSM solution was 51.0 wt. %, the Alkanol-XC solution was 2.0 wt. %, the FT-248 solution was 0.3 wt. %, and the water was 35.45 wt. %. The thickness of the dry layer was measured to be about 1.8±0.1 μm.

### Example 3

This example illustrates the method of evaluating gelatin-coated biological, here, protein, microarray substrate using a modified Enzyme Linked Immunosobent Assay (ELISA). This example also illustrates the reduction of background fluorescence by incorporating filler particles into gelatin binder to produce a filled gelatin layer.

The procedure to perform the modified ELISA follows.

1. Goat anti-mouse antibody IgG from Sigma was dissolved in PBS (phosphate saline buffer, pH7.4) buffer to a concentration of 1 mg/mL. A series of diluted of goat anti-mouse antibody IgG were spotted onto gelatin coated substrates using a Cartisian Arrayer. The spotted substrates were incubated in a humid chamber for 1 hour at room temperature.

2. The substrates were washed four times in PBS buffer with 1% nonionic surfactant Triton X100™, 5 min each time with shaking.

3. The washed substrates were incubated in PBS buffer with 1% glycine for 15 min with constant shaking.

4. The substrates were washed four times in PBS buffer with 1% Triton™ X100 with shaking.

5. Mouse IgG from Sigma was diluted in PBS buffer with 0.1% nonionic surfactant Tween™ 20 to 1 μg/mL to cover the whole surface of substrates, and the substrates were incubate at room temperature for 1 hour.

6. The substrates were washed four times with PBS buffer with 1% Triton X100, 5 min each time with constant shaking.

7. The substrates were incubated in goat anti-mouse IgG horse radish peroxidase conjugate (diluted in PBS with 1% glycine to appropriate titer) solution to cover the whole surface of the substrates at room temperature for 1 hour with shaking.

8. The substrates were washed four times with PBS buffer with 1% Triton X100, 5 min each time with constant shaking, and rinsed twice in water.

The signals were developed in horse radish peroxidase substrate solution containing SuperSignal® ELISA chemiluminescence substrate solution (purchased from PIERCE ENDOGEN). The chemiluminescence image was captured by contacting a thin layer of SuperSignal® ELISA chemiluminescence substrate solution (purchased from PIERCE ENDOGEN) with coated substrate. The emission was measured on Kodak Image Station 440 and quantified using Region of Interest (ROI) software supplied with the instrument. The results are summarized in Table 1 and Table 2.

The fluorescence of the coated glass slides in Table 1 were obtained on the JY LabRam instrument, excited by 272 mW of 514.5 nm laser light that was incident on a 2 OD neutral density filter. The laser light was focused onto the sample through a 100×short-working-distance microscope objective. The scattered light (including fluorescence) was collected by the same objective and spatially filtered by a 400 micron confocal hole, before incidence on a 250 micron slit. The fluorescence signal was acquired for 500 seconds in duplicate acquisitions. Two spots at opposite ends of each slide were measured and the fluorescence at 570 nm was reported in Table 1.

The fluorescence of the coated glass slides in Table 2 were obtained on a JY LabRam instrument, excited by 195 mW of 514.5 nm laser light that was incident on a 2 optical density neutral density filter. The laser light was focused onto the sample through a 50×short-working-distance microscope objective. The scattered light (including fluorescence) was collected by the same objective and spatially filtered by a 835 micron confocal hole, before incidence on a 250 micron slit. The fluorescence signal was acquired for 60 seconds in duplicate acquisitions. Two spots on each slide were measured and the fluorescence at 570 nm was summarized in Table 2.

TABLE 1
Performance evaluation for coatings containing
inorganic filler particles

| | | | Antibody | Antibody | Antibody |
|---|---|---|---|---|---|
| | Coating | | printed | printed | printed |
| Element | thickness | Fluorescence | 1 ng | 0.5 ng | 0.1 ng |
| 1 | 1.40 | 479 | 97228 | 65377 | 9808 |
| 2 | 1.40 | 787 | 53638 | 43069 | 10773 |
| 3 | 0.90 | 542.5 | 48582 | 42090 | 10388 |
| 4 | 2.00 | 690 | 56959 | 50751 | 9747 |
| 5 | 1.80 | 588.5 | 54513 | 32476 | 7404 |
| Control 6 | 1.70 | 3176 | 25850 | 24019 | 4600 |

TABLE 2
Performance evaluation for coatings containing
organic filler particles

| | | | Antibody | Antibody | Antibody |
|---|---|---|---|---|---|
| | Coating | | printed | printed | printed |
| Element | thickness | Fluorescence | 1 ng | 0.5 ng | 0.1 ng |
| 7 | 1.8 | 1011 | 27796 | 23423 | 9038 |
| 8 | 0.6 | 431 | 56228 | 40094 | 10993 |
| 9 | 3.2 | 919.0 | 57732 | 47846 | 14012 |
| 10 | 1.7 | 1123 | 41971 | 21756 | 2896 |
| 11 | 1.8 | 847 | 25463 | 13953 | 2628 |
| Control 12 | 1.8 | 1459 | 23828 | 24185 | 1398 |

The results in Table 1 and 2 indicate that, in comparison to the control sample, the gelatin coatings with filler particles incorporated in the gelatin layer provide a substrate with much reduced fluorescence background as well as improved antibody binding capacity.

The invention has been described in detail with particular reference to certain preferred embodiments thereof, but it will be understood that variations and modifications can be effected within the spirit and scope of the invention.

1. A biological microarray comprising a support having disposed thereon at least one layer comprising filler and gelatin, and at least one functional compound, wherein said functional compound comprises a first functional group capable of interacting with said gelatin and a second functional group capable of interacting with a biological capture agent, wherein said first functional group is the same as or different from the second functional group.

2. The biological microarray of claim 1 wherein said biological microarray has reduced background noise.

3. The biological microarray of claim 1 wherein said support comprises a support having dimensional stability.

4. The biological microarray of claim 1 wherein said support comprises glass.

5. The biological microarray of claim 1 wherein said support comprises a polymer.

6. The biological microarray of claim 5 wherein said polymer support has been surface treated to enhance adhesion of said at least one layer comprising filler and gelatin to said support.

7. The biological microarray of claim 1 wherein said gelatin comprises alkaline pretreated gelatin.

8. The biological microarray of claim 1 wherein said gelatin comprises bovine gelatin, pig gelatin, fish gelatin or fowl gelatin.

9. The biological microarray of claim 1 wherein said gelatin is present in an amount of from 15 to 99% by weight of said layer.

10. The biological microarray of claim 1 wherein said filler comprises a mean particle diameter of less than 1.0 micrometers.

11. The biological microarray of claim 1 wherein said filler comprises from 1 to 85% by weight of said layer.

12. The biological microarray of claim 1 wherein said filler comprises inorganic particles, organic particles, soluble polymers or combinations thereof.

13. The biological microarray of claim 12 wherein said organic particles comprise polymer particles.

14. The biological microarray of claim 13 wherein said polymer particles comprise water insoluble synthetic polymers.

15. The biological microarray of claim 14 wherein said water insoluble synthetic polymers comprise at least one member selected from the group consisting of addition polymers, poly (alkylene oxides), phenol-formaldehyde polymers, urea-formaldehyde polymers and condensation polymers consisting of one or more of the following repetitive units: esters, amides, imides, carbonates, urethanes, and ethers.

16. The biological microarray of claim 13 wherein said polymer particles comprise monodisperse polymer particles.

17. The biological microarray of claim 16 wherein said monodisperse polymer particles have a particle size distribution, wherein the coefficient of said particle size distribution is less than 20%.

18. The biological microarray of claim 13 wherein said polymer particles comprise polymers made from monomers containing α,β-ethylenic unsaturation.

19. The biological microarray of claim 18 wherein said monomers have limited solubility in water.

20. The biological microarray of claim 18 wherein said monomers comprise at least one member selected from the group consisting of styrenics, acrylic esters, methacrylic esters, acrylamides, methacrylamides, or vinyl esters.

21. The biological microarray of claim 18 wherein said monomers comprise aliphatic acrylic esters or methacrylic esters.

22. The biological microarray of claim 18 wherein said monomers comprise at least two ethylenically unsaturated chemical functionalities.

23. The biological microarray of claim 22 wherein said monomers comprise at least one member selected from the group consisting of vinyl groups, acrylates, methacrylates, vinyl ethers and vinyl esters.

24. The biological microarray of claim 18 further comprising less than 20% of the total weight of the polymerizeable solids of one or more water-soluble ethylenically unsaturated monomers.

25. The biological microarray of claim 13 wherein said polymer particles comprise chemically active groups.

26. The biological microarray of claim 25 wherein said chemically active groups comprise at least one member selected from the group consisting of thiols, primary amines, secondary amines, tertiary amines, quaternary ammoniums, phosphines, alcohols, carboxylic acids, primary or secondary amines, vinylsuflonyls, aldehydes, epoxies, hydrazides, succinimidyl esters, carbodiimides, maleimides, iodoacetyls, isocyanates, isothiocyanates, aziridines, or sulfonates.

27. The biological microarray of claim 12 wherein said soluble polymers comprise linear or branched soluble polymers.

28. The biological microarray of claim 12 wherein said soluble polymers comprise polymers soluble in water or water-miscible solvents.

29. The biological microarray of claim 28 wherein said soluble polymers comprise at least one member selected from the group consisting of methanol, ethanol, isopropanol, 1-methoxy-2-propanol and n-propanol), methyl ethyl ketone, tetrahydrofuran, acetonitrile and acetone.

30. The biological microarray of claim 12 wherein said soluble polymers comprise natural soluble polymers.

31. The biological microarray of claim 30 wherein said soluble polymers comprise polysaccharides.

32. The biological microarray of claim 12 wherein said soluble polymers comprise synthetic soluble polymers.

33. The biological microarray of claim 32 wherein said soluble polymers comprise contain greater than 1.7 mEq/g of amide, amine, and heterocyclic nitrogen groups.

34. The biological microarray of claim 12 wherein said inorganic particles comprise inorganic oxide or inorganic powder.

35. The biological microarray of claim 34 wherein said inorganic particles at least one member selected from the group consisting of silica, colloidal silica, silicon oxide dispersions, fumed silica, aluminum oxide, colloidal alumina, fumed alumina, calcium carbonate, kaolin, talc, calcium sulfate, natural or synthetic clay, barium sulfate, barium sulfate mixtures with zinc sulfide, γ-aluminum oxide, chromium oxide, iron oxide, tin oxide, doped tin oxide, alumino-silicate, titanium dioxide, silicon carbide, titanium carbide, diamond in fine powder, titanium dioxide, zinc oxide, or mixtures thereof.

36. The biological microarray of claim 34 wherein said inorganic particles colloidal particles.

37. The biological microarray of claim 1 wherein said functional compound comprises at least a first functional group A capable of interacting with said gelatin and at least a second functional group B capable of interacting with a biological capture agent, wherein said first functional group A is the same as or different from the second functional group B, and wherein said first functional group A is connected to said second functional group B by a linking group L, wherein said L is capable of interacting with said A and with said B.

38. The biological microarray of claim 37 wherein A and B each independently comprise a member from the group consisting of aldehyde, epoxy, hydrazide, vinyl sulfone, succinimidyl ester, carbodiimide, maleimide, dithio, iodoacetyl, isocyanate, isothiocyanate, or aziridine.

39. The biological microarray of claim 37 wherein B comprises at least one member selected from the group consisting of streptavidin, biotin, glutathione-S-transferase, glutathione, or histidine.

40. The biological microarray of claim 37 wherein L comprises a diradical of such a length that the shortest through-bond path between the ends that connect A to B is not greater than 10 atoms.

41. The biological microarray of claim 37 wherein L comprises a single bond, a carbon atom, an oxygen atom, a sulfur atom, a carbonyl group, a carboxylic ester group, a carboxylic amide group, a sulfonyl group, a sulfonamide group, an ethyleneoxy group, a polyethyleneoxy group, or an amino group.

42. The biological microarray of claim 37 wherein L further comprises solubilizing groups selected from the group consisting of carboxylic acid, sulfonic acid, phosphonic acid, hydroxamic acid, sulfonamide, and hydroxy groups (and their corresponding salts).

43. The biological microarray of claim 1 wherein said functional polymer compound is represented by Formula I: 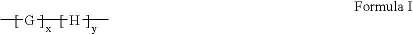
wherein
“G” represents a polymerized α,β-ethylenically unsaturated addition polymerizeable monomer;
“H” represents a vinylsulfone or vinylsulfone precursor unit monomer; and
x and y both represent molar percentages ranging from 10 to 90 and 90 to 10.

44. The biological microarray of claim 43 wherein G represents nonionic or ionic monomers.

45. The biological microarray of claim 44 wherein said ionic monomers comprise at least one member selected from the group consisting of 2-phosphatoethyl acrylate potassium salt, 3-phosphatopropyl methacrylate ammonium salt, acrylamide, methacrylamides, maleic acid and salts thereof, sulfopropyl acrylate and methacrylate, acrylic and methacrylic acids and salts thereof, N-vinylpyrrolidone, acrylic and methacrylic esters of alkylphosphonates, styrenics, acrylic and methacrylic monomers containing amine ammonium functionalities, styrenesulfonic acid and salts thereof, acrylic and methacrylic esters of alkylsulfonates, vinylsulfonic acid and salts thereof.

46. The biological microarray of claim 44 wherein said nonionic monomers comprise at least one member selected from the group consisting of poly(ethylene oxide) segments, carbohydrates, amines, amides, alcohols, polyols, nitrogen-containing heterocycles, and oligopeptides.

47. The biological microarray of claim 44 wherein said nonionic monomers comprise at least one member selected from the group consisting of poly(ethylene oxide) acrylate and methacrylate esters, vinylpyridines, hydroxyethyl acrylate, glycerol acrylate and methacrylate esters, (meth)acrylamide, and N-vinylpyrrolidone.

48. The biological microarray of claim 43 wherein G represents the polymerized form of acrylamide, sodium 2-acrylamido-2-methanepropionate, sulfopropyl acrylate and methacrylate salts, or sodium styrenesulfonate.

49. The biological microarray of claim 43 wherein H represents the polymerized form of a vinylsulfone or vinylsulfone precursor unit.

50. The biological microarray of claim 43 wherein said “H” represents groups represented by Formula II: 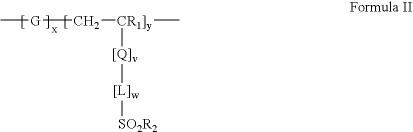
wherein:
R₁ is a hydrogen atom or a C₁-C₆ alkyl group;
Q is —CO₂—, or CONR₁.;
v is 1 or 0;
w is 1-3;
L is a divalent linking group containing at least one linkage selected from the group consisting of —CO₂— and —CONR₁, and containing 3-15 carbon atoms, or a divalent atom containing at least one linkage selected from the group consisting of —O—, —N(R₁)—, —CO—, —SO—, —SO₂—, —SO₃—, —SO₂N(R₁)—, —N(R₁)CON(R₁)— and —N(R₁)CO₂—, and containing 1-12 carbon atoms in which R₁ has the same meaning as defined above;
R₂ is —CH═CH2 or —CH2-CH2X₁ wherein X1 is a substituent replaceable by a nucleophilic group or releasable in the form of HX, by a base.

51. The biological microarray of claim 1 wherein said second functional group is capable of interacting non-covalently with a biological capture agent.

52. The biological microarray method of claim 1 wherein said functional compound is disposed in said at least one layer comprising filler and gelatin.

53. The biological microarray method of claim 1 wherein said functional compound is disposed onto said at least one layer comprising filler and gelatin.

54. The biological microarray of claim 1 wherein said interacting with said gelatin comprises a physical binding or a chemical reaction.

55. The biological microarray of claim 1 wherein said interacting with said biological capture agent comprises a physical binding or a chemical reaction.

56. The biological microarray of claim 1 further comprising a biological capture agent.

57. The biological microarray of claim 56 wherein said biological capture agent comprises at least one member selected from the group consisting of antibodies, proteins, polymer scaffolds, peptides, antigens, nucleic acid ligands, or molecular imprinting polymers.

58. The biological microarray of claim 57 wherein said polymer scaffold comprises at least one polymer rich in reactive units capable of immobilizing biological compounds.

59. The biological microarray of claim 58 wherein said reactive unit comprises at least one member selected from the group consisting of aldehyde, epoxy, hydrazide, vinyl sulfone, succinimidyl ester, carbodiimide, maleimide, dithio, iodoacetyl, isocyanate, isothiocyanate, or aziridine.

60. The biological microarray of claim 58 wherein said at least one polymer comprises poly(vinylamine), poly(propyleneimine), poly(N-aminopropyl methacrylamide) or poly(n-vinylimidazole).

61. The biological microarray of claim 57 wherein said polymer scaffold comprises at least one precursor polymer.

62. The biological microarray of claim 61 wherein said precursor polymer is rich in thiols, amines, phosphines, alcohols, or carboxylic acids.

63. The biological microarray of claim 61 wherein the precursor polymer is rich in primary or secondary amines.

64. The microarray of claim 1 further comprising at least one interlayer between said at least one layer and said support.

65. The microarray of claim 1 further comprising additives.

66. A method of making a biological microarray element comprising:
providing a support; and
coating a layer comprising filler and gelatin and at least one functional compound, wherein said functional compound comprises a first functional group capable of interacting with said gelatin and a second functional group capable of interacting with a biological capture agent, wherein said first functional group is the same as or different from the second functional group.

67. The method of claim 66 wherein said coating comprises simultaneous coating.

68. The method of claim 66 wherein said coating comprises sequential coating.

69. The method of claim 66 further comprising modifying said support prior to coating said layer comprising filler and gelatin and said at least one functional compound.
